(19) United States
(12) Patent Application Publication
(10) Pub. No.: US 2023/0173179 A1
(43) Pub. Date: Jun. 8, 2023

(54) Injection Device

(71) Applicant: Sanofi, Paris (FR)

(72) Inventors: Stephan Riedel, Frankfurt am Main (DE); Stefan Alt, Frankfurt am Main (DE)

(21) Appl. No.: 17/922,702

(22) PCT Filed: May 11, 2021
(86) PCT No.: PCT/EP2021/062401

§ 371 Date: Nov. 1, 2022

(30) Foreign Application Priority Data
May 11, 2020 (EP) 20315238.4

(51) Int. Cl. A61M 5/20; A61M 5/315

(52) U.S. Cl. CPC A61M 5/20; A61M 5/315; A61M 5/31545

(57) ABSTRACT

An injection device includes a housing; an actuation member movable between a first position relative to the housing and a second position relative to the housing, wherein the actuation member is arranged to inhibit a mechanical operation of the injection device when the actuation member is in the first position, and allow the mechanical operation of the injection device when the actuation member is in the second position; electronic circuitry; and an actuation sensing arrangement, wherein the actuation sensing arrangement is configured to detect movement of the actuation member from the first position relative to the housing to the second position relative to the housing, and wherein the electronic circuitry is configured to be activated in response to detection by the actuation sensing arrangement that the actuation member has moved from the first position relative to the housing to the second position relative to the housing.

### CROSS-REFERENCE TO RELATED APPLICATIONS

The present application is the national stage entry of International Patent Application No. PCT/EP2021/062401, filed on May 11, 2021, and claims priority to Application No. 20315238.4, filed on May 11, 2020, the disclosures of which are incorporated herein by reference.

### TECHNICAL FIELD

The present disclosure relates to an injection device, in particular an injection device that can be activated.

### BACKGROUND

Needle based injection systems (NIS) for the self-administration of drugs such as insulin by patients can be found in a convenient pen shape (i.e. insulin injection pens). In many examples of injection pens, drug delivery is a purely mechanical operation. However, recent variants of these injection pens include electronic components that may perform a number of functions such as dose measurement, data storage and data transmission. These electronic components require a power supply to function. However compact nature of many injection devices can place a constraint on the size of battery that can be accommodated in the injection device.

### SUMMARY

According to an aspect of the present disclosure, there is provided an injection device comprising: a housing; an actuation member movable between a first position relative to the housing and a second position relative to the housing, wherein the actuation member is arranged to inhibit a mechanical operation of the injection device when the actuation member is in the first position, but allow the mechanical operation of the injection device when the actuation member is in the second position; electronic circuitry; and an actuation sensing arrangement, wherein the actuation sensing arrangement is configured to detect movement of the actuation member from the first position relative to the housing to the second position relative to the housing, and wherein the electronic circuitry is configured to be activated in response to detection by the actuation sensing arrangement that the actuation member has moved from the first position relative to the housing to the second position relative to the housing.

The mechanical operation may comprise a dose programming operation.

The mechanical operation may comprise a dose dispensing operation.

Activation of the electronic circuitry may comprise switching the electronic circuitry from a relatively low-power state to a relatively high-power state.

The actuation member may comprise a hinged member pivotally coupled to the housing such that the hinge member can pivot between the first position and the second position relative to the housing.

The hinged member may be configured to engage with a mechanical part of the injection device to inhibit movement of the mechanical part when the hinged member is in the first position. The hinged member may be disengaged with the mechanical part when in the second position.

The actuation sensing arrangement may comprise a plurality of conductive contacts; wherein the hinged member comprises an electrically conductive portion, wherein the conductive contacts and the electrically conductive portion are arranged such that: the electrically conductive portion forms an electrical connection between the conductive contacts when the actuation member is in one of the first position and the second position; and there is no electrical connection between the conductive contacts via the electrically conductive portion when the actuation member is in the other one of the first position and the second position.

The actuation sensing arrangement may comprise a hinge switch arranged to detect whether the hinged member is in the first position or the second position.

The actuation sensing arrangement may comprise a reed switch; and wherein the hinged member comprises a magnet, wherein the magnet and the reed switch are arranged such that the reed switch is in a first switch state when the actuation member is in the first position and the reed switch is in a second switch state when the actuation member is in the second position.

The injection device may further comprise a dosage knob; wherein the dosage knob is biased from a first linear position relative to the housing towards a second linear position relative to the housing; wherein the hinged member is configured to inhibit movement of the dosage knob from the first linear position to the second linear position when the hinged member is in the first position, wherein the dosage knob is configured to move from the first linear position to the second linear position in response to the hinged member being moved from the first position to the second position, wherein the actuation sensing arrangement is configured to detect movement of the dosage knob from the first linear position relative to the housing to the second linear position relative to the housing, and wherein the electronic circuitry is configured to be activated in response to the detection by the actuation sensing arrangement that the dosage knob has moved from the first linear position relative to the housing to the second linear position relative to the housing.

The actuation member may comprise a container configured to hold the injection device.

The actuation member may comprise a container configured to hold the injection device, wherein the actuation sensing arrangement is configured to detect removal of the injection device from the container, and wherein the electronic circuitry is configured to be activated in response to detection by the actuation sensing arrangement that the injection device has been removed from the container.

The actuation member may comprise a collar. The collar may be movable from the first position relative to the housing to the second position relative to the housing. For example, the collar may be movable along the housing from the first position relative to the housing to the second position relative to the housing. The collar may be threadingly engaged with the housing of the injection device.

The actuation member may comprise a collar threadingly engaged with the housing of the injection device, wherein the collar is movable along the housing from the first position relative to the housing to the second position relative to the housing, wherein the actuation sensing arrangement is configured to detect movement of the collar from the first position relative to the housing to the second position relative to the housing, and wherein the electronic circuitry is configured to be activated in response to detection by the actuation sensing arrangement that the collar has moved from the first position relative to the housing to the second position relative to the housing.

The actuation sensing arrangement may be further configured to detect movement of the actuation member from the second position relative to the housing to the second position relative to the housing, and the electronic circuitry may be configured to be switched from an activated state to a dormant state in response to the detection by the actuation sensing arrangement that the actuation member has moved from the second position relative to the housing to the first position relative to the housing.

The injection device may further comprise a container containing a medicament.

The injection device may be an injection pen or a patch pump.

The injection device may comprise a dosage knob (and/or dose dialling member) biased from a first position relative to the housing to a second position relative to the housing. The first position may be a first linear position relative to the housing and the second position may be a second linear position relative to the housing. The actuation member may be configured to inhibit movement of the dosage knob (and/or dose dialling member) from the first position of the dosage knob (and/or dose dialling member) to the second position of the dosage knob (and/or dose dialling member) when the actuation member is in the first position of the actuation member. The actuation member may be configured to not inhibit movement of the dosage knob (and/or dose dialling member) from the first position of the dosage knob (and/or dose dialling member) to the second position of the dosage knob (and/or dose dialling member) when the actuation member is in the second position of the actuation member. The dosage knob (and/or dose dialling member) may be configured to move from the first position of the dosage knob (and/or dose dialling member) to the second position of the dosage knob (and/or dose dialling member) in response to the actuation member being moved from the first position of the actuation member to the second position of the actuation member. The actuation sensing arrangement may be configured to detect movement of the dosage knob (and/or dose dialling member) from the first position relative to the housing to the second position relative to the housing, and the electronic circuitry may be configured to be activated in response to the detection by the actuation sensing arrangement that the dosage knob (and/or dose dialling member) has moved from the first position relative to the housing to the second position relative to the housing.

According to another aspect, there is provided a method of activating an injection device disclosed herein, the method comprising: detecting movement of the actuation member from a first position relative to the housing to a second position relative to the housing, and activating the electronic circuitry in response to detection that the actuation member has moved from the first position relative to the housing to the second position relative to the housing.

Aspects of the present disclosure may better conserve energy stored in the power supply of an injection device. Aspects of the present disclosure may also provide protection against accidental activation of the injection device, such as during handling, transport and storage of the injection device.

### BRIEF DESCRIPTION OF THE FIGURES

Exemplary embodiments of the present disclosure are described with reference to the accompanying drawings, in which:

FIG. 1 is a side-on view of an injection device;

FIG. 2 is a schematic diagram of an electronic circuit within the injection device of FIG. 1;

FIG. 3A is a side-on view of an injection device according to a first embodiment;

FIG. 3B is a side-on view of the injection device of FIG. 3A with the actuation member in a different position;

FIG. 4 is a schematic partial cross-section of the injection device of FIG. 3A, with the hinged member in a first position;

FIG. 5 is a schematic partial cross-section of the injection device of FIG. 4, with the hinged member in a second position;

FIG. 6 is a schematic partial side-view of an injection device according to a second embodiment, with the hinged member in a first position;

FIG. 7 is a schematic partial side-view of the injection device of FIG. 6, with the hinged member in a second position;

FIG. 8 is a schematic partial side-view of an injection device according to a third embodiment, with the hinged member in a first position;

FIG. 9 is a schematic partial side-view of the injection device of FIG. 8, with the hinged member in a second position;

FIG. 10 is a schematic partial side-view of an injection device according to a fourth embodiment, with the hinged member in a first position;

FIG. 11 is a schematic partial side-view of the injection device of FIG. 10, with the hinged member in a second position;

FIG. 12 is a schematic partial side-view of an injection device according to a fifth embodiment, with the collar in a first position;

FIG. 13 is a schematic partial cross-section of the injection device of FIG. 12;

FIG. 14 is a schematic partial side-view of the injection device of FIG. 12, with the collar in a second position;

FIG. 15 is a schematic partial cross-section of the injection device of FIG. 14;

FIG. 16A is a schematic side-view of an injection device according to a sixth embodiment, with the injection device held in a container;

FIG. 16B is a schematic partial side-view of the injection device of FIG. 16A, with the injection device partially removed from the container;

FIG. 17 is a flowchart illustrating a method according to another aspect of the disclosure;

FIG. 18 is a flowchart illustrating a method according to another aspect of the disclosure.

Reference will now be made in detail to the embodiments of the present disclosure, examples of which are illustrated in the accompanying drawings, wherein like reference numerals refer to like elements throughout.

### DETAILED DESCRIPTION

In the following, embodiments of the present disclosure may be described with reference to an insulin injection device. The present disclosure is however not limited to such application and may equally well be deployed with injection devices that eject other medicaments.

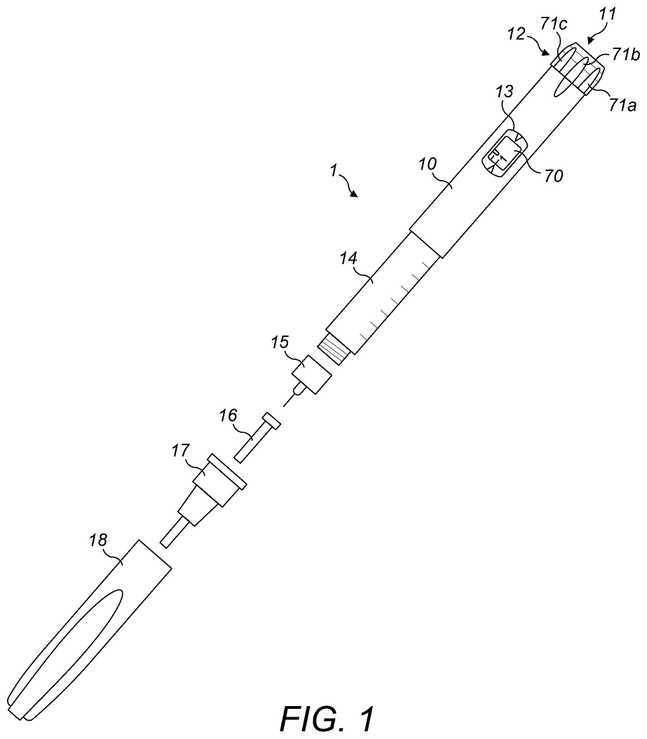

FIG. 1 is an exploded view of a medicament delivery device. In this example, the medicament delivery device is an injection device 1, such as Sanofi's SoloSTAR® insulin injection pen. However, aspects of the disclosure may apply to other types of injection pens or injection devices. Aspects of the present disclosure may apply to an injection device 1 taking the form of autoinjectors or patch pumps. Aspects of the present disclosure may apply to an injection device 1 suitable for one-time use or for repeated use.

The injection device 1 of FIG. 1 is a pre-filled, disposable injection pen that comprises a cylindrical housing 10 and contains an insulin container 14, to which a needle 15 can be affixed. The needle is affixed at the distal end of the housing 10. The term “distal” as used throughout refers to a location that is relatively closer to a site of injection, and the term “proximal” refers to a location that is relatively further away from the injection site. The needle 15 is protected by an inner needle cap 16 and either an outer needle cap 17 or other cap 18.

An insulin dose to be ejected from injection device 1 can be programmed, or ‘dialled in’ by turning a dosage knob 12, and a currently programmed dose is then displayed via dosage window 13, for instance in multiples of units. For example, where the injection device 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a number sleeve 70 that is configured to move when the dosage knob 12 is turned, to provide a visual indication of a currently programmed dose. The dosage knob 12 is rotated on a helical path with respect to the housing 10 when turned during programming.

In this example, the dosage knob 12 includes one or more formations 71a, 71b, 71c to assist a user in gripping the dosage number 12 during programming. The formations 71a, 71b, 71c may be grooves, ridges or the like.

The injection device 1 may be configured so that turning the dosage knob 12 causes a mechanical click sound to provide acoustical feedback to a user. The number sleeve 70 mechanically interacts with a piston in the insulin container 14. When the needle 15 is stuck into a skin portion of a patient, and then the injection button 11 is pushed, the insulin dose displayed in the display window 13 will be ejected from the injection device 1. When the needle 15 of the injection device 1 remains for a certain time in the skin portion after the injection button 11 is pushed, a high percentage of the dose is actually injected into the patient's body. Ejection of the insulin dose may also cause a mechanical click sound, which is however different from the sounds produced when using dosage knob 12.

In this example, during delivery of the insulin dose, the dosage knob 12 is turned to its initial position in an axial movement, that is to say without rotation, while the number sleeve 70 is rotated to return to its initial position, e.g. to display a dose of zero units.

The injection device 1 may be used for several injection processes until either the insulin container 14 is empty or the expiration date of the medicament in the injection device 1 (e.g. 28 days after the first use) is reached.

Furthermore, before using the injection device 1 for the first time, it may be necessary to perform a so-called “prime shot” to remove air from the insulin container 14 and the needle 15, for instance by selecting two units of insulin and pressing the injection button 11 while holding the injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user. Nevertheless, differences (e.g. losses) between the ejected amounts and the injected doses may need to be taken into account.

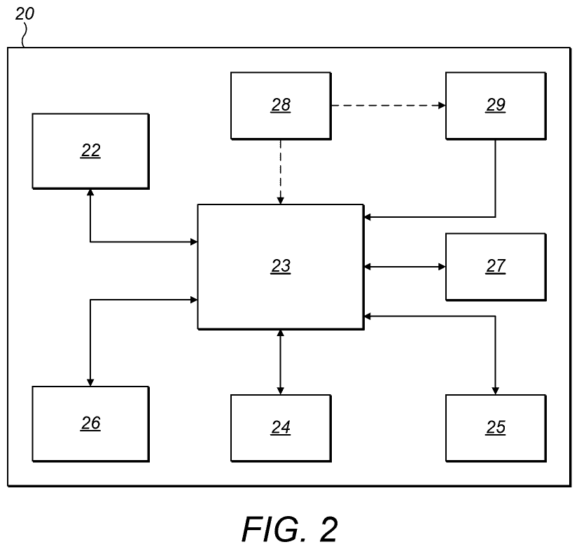

In accordance with embodiments of the disclosure, the injection device 1 further contains electronic circuitry 20 as schematically illustrated in FIG. 2.

The electronic circuitry 20 may be configured for performing one or more functions of the injection device 1. For example, the one or more functions may include one or more of a monitoring function for one or more operations or variables associated with the injection device, such as one or more operations related to programming of a dose or dispensing of a dose. For example, the electronic circuitry 20 may be configured to determine at least one of a dose dialled into the injection device 1, a dose dispensed from the injection device 1, a time and/or date of dose dialling and/or dose dispensing, whether an operation is a priming operation or a dose dispensing operation, or a temperature of the injection device 1.

As shown in FIG. 2, the electronic circuitry 20 includes a processor arrangement 23 including one or more processors, such as a microprocessor, a Digital Signal Processor (DSP), Application Specific Integrated Circuit (ASIC), Field Programmable Gate Array (FPGA) or the like, together with one or more memory units 24, 25, such as program memory 24 and main memory 25, which can store software for execution by the processor arrangement 23 and data collected by the injection device 1.

A communication interface 27 may be provided, which may be a wireless communications interface for communicating with another device via a wireless network such as Wi-Fi, Bluetooth®, NFC or the like, or an interface for a wired communications link, such as a socket for receiving a Universal Series Bus (USB), mini-USB or micro-USB connector. The other device may be a mobile computing device such as a smartphone. The processor arrangement 23 may be configured to transmit data collected by the injection device 1 to the other device via the communication interface 27. For example, the processor arrangement 23 may be configured to transmit data indicative of one or more doses dispensed by the injection device 1 to the other device via the communication interface 27. The other device may store and/or further process the data received from the injection device 1. In some examples, the processor arrangement 23 may be configured to transmit data to a server and/or cloud via the communication interface 27, for storage or processing of the data.

The electronic circuitry 20 may optionally comprise a display 22. The display 22 is located on the injection device 1 so that it can provide a visual output to a user. The display 22 may comprise one or more LEDs, an LCD display, or any other suitable display means. The display 22 is controlled by the processor arrangement 23 to provide the visual output. The visual output may indicate to a user a status of the injection device 1, such as whether the injection device 1 is switched on or ready for an injection. The visual output may indicate to a user a dose value such as a dose dialled into the injection device 1 or a dose dispensed from the injection device 1. The visual output may indicate instructions to the user regarding how or when to perform various operations using the injection device 1, for example when to perform a priming operation. In some examples, the electronic circuitry 20 may comprise an audio transducer and/or haptic transducer (not shown) in addition or alternative to the display 22. The audio transducer and/or haptic transducer is controlled by the processor arrangement 23 to provide an audio or haptic output to a user.

A power source 29 such as a battery is provided. The power source 29 provides electrical power to one or more components of the electronic circuitry 20.

A sensor arrangement 26 may be provided. The sensor arrangement 26 may comprise one or more sensors configured to detect or determine one or more properties associated with the injection device 1. For example, the sensor arrangement 26 may comprise a dose determination unit configured to determine a dose programmed into the injection device 1 by a user, and/or a dose dispensed by the injection device 1 during an injection operation. In some examples, the sensor arrangement 26 may be configured to detect one or more of an actuation of the injection button 11 by a user, a replacement of a medicament container 14, a removal or replacement of a cap 18, or the like.

The electronic circuitry 20 further comprises an actuation sensing arrangement 28. The actuation sensing arrangement 28 is configured determine whether an actuation member of the injection device 1 is in a first position or a second position with respect to the housing 10 of the injection device 1. This is described in more detail in the embodiments below. The actuation sensing arrangement 28 provides a different output depending on whether the actuation member of the injection device 1 is in the first position or the second position.

The actuation member is arranged to inhibit a mechanical operation of the injection device 1 when the actuation member is in the first position, but allow the mechanical operation of the injection device 1 when the actuation member is in the second position. A user moves the actuation member from the first position to the second position so that the mechanical operation may take place.

The mechanical operation may be a dose programming operation, also known as a dose setting operation. The presence of the actuation member in the first position will prevent the dose programming operation from being performed by a user. For example, the presence of the actuation member in the first position may inhibit a dose being dialled into the injection device 1 using the dosage knob 12. The actuation member when in the first position may inhibit at least one of the dosage knob 12 being rotated or translated by a user relative to the housing 10, or the dose dialling member 19 being rotated or translated by a user relative to the housing 10. The user moves the actuation member from the first position to the second position in order to allow the dose programming operation to take place. The presence of the actuation member in the second position may allow a dose to be dialled into the injection device 1 using the dosage knob 12. The actuation member when in the second position may allow at least one of the dosage knob 12 being rotated or translated by a user relative to the housing 10, or the dose dialling member 19 being rotated or translated by a user relative to the housing 10.

The mechanical operation may be a dose dispensing operation, in which case the presence of the actuation member in the first position will prevent the dose dispensing operation from being performed by a user. For example, the presence of the actuation member in the first position may inhibit actuation of the injection button 11 by a user relative to the housing. The presence of the actuation member in the first position may inhibit translation or rotation of the dosage knob 12 relative to the housing 10, which would otherwise have occurred during a dose dispensing operation. The presence of the actuation member in the first position may inhibit translation or rotation of the dose dialling member 19 relative to the housing 10, which would otherwise have occurred during a dose dispensing operation. The user moves the actuation member from the first position to the second position in order to allow the dose dispensing operation to take place. The presence of the actuation member in the second position may allow a dose to be dispensed by the injection device 1. The actuation member when in the second position may allow actuation of the injection button 11 by a user relative to the housing, allow translation or rotation of the dosage knob 12 relative to the housing 10, or allow translation or rotation of the dose dialling member 19 relative to the housing 10.

The actuation sensing arrangement 28 is used to activate the injection device 1. A user activates the injection device 1 by moving the actuation member from the first position to the second position with respect to the housing 10. The injection device 1 is activated in response to a detection by the actuation sensing arrangement 28 that the actuation member has moved from the first position to the second position. In particular, it is the electronic circuitry 20 of the injection device 1 that is activated in response to the detection that the actuation member has moved from the first position to the second position.

Activation of the electronic circuitry 20 can mean switching on power to one or more components of the electronic circuitry 20, or can mean waking one or more components of the electronic circuitry 20 from a low-power state to a higher-power state. Before the electronic circuitry 20 is activated, it is said to be in a dormant or inactivated state. In the dormant state, one or more components of the electronic circuitry 20 receive no power from the power supply 29 or are in a low-powered state relative to when the electronic circuitry 20 is activated.

In a dormant state of the electronic circuitry 20, one or more sensors of the sensor arrangement 26 may be cut off from power from the power source 29 or may be in a low-powered state. In the activated state of the electronic circuitry 20, the one or more previously dormant sensors of the sensor arrangement 26 may now have power from the power source 29 supplied to them, or may now be switched to a relatively higher-powered state.

In some examples, the processor arrangement 23 may be cut off from power from the power source 29 or may be in a low-powered state (e.g. standby/shutdown state) when the electronic circuitry 20 is in the dormant state. Once the electronic circuitry 20 has been activated, the processor arrangement 23 may now receive power from the power source 29, or may now be in a relatively higher-powered state compared to the previous low-powered state.

Activating the electronic circuitry 20 may comprise switching the electronic circuitry 20 from an ‘OFF’ state or a ‘STANDBY’ state to an ‘ON’ state. Activating the electronic circuitry 20 may therefore comprise connecting the electronic circuitry 20 to the power supply 29.

The actuation member may be a catch, a latch, a lock, a clamp or the like. The actuation member can prevent a mechanical operation of the injection device 1 from taking place, such as a dose programming operation or a dose dispensing operation. The actuation member may prevent the mechanical operation from accidentally being started, for example accidentally being started during storage or transportation of the injection device 1. The actuation member may also prevent the mechanical operation from intentionally being started by certain users, such as children.

The presence of the actuation member can also inhibit accidental activation of the electronic circuitry 20, for example during storage or transportation of the injection device 1. This may reduce the likelihood of the power source 29 of the injection device 1 being accidentally drained.

A user moves the actuation member in order to perform an important mechanical operation such as a dose programming or dispensing operation. Activation of the electronic circuitry 20 may therefore be intuitive to a user, since the activation is performed in response to a user performing an action that they perform to administer a dose. The actuation member may serve a dual purpose, acting as both a safety lock for the injection device 1 and as a means for activating the electronic circuitry 20. This may reduce the number of steps that are performed by a user when administering a dose using the injection device 1. Operation of the injection device 1 may be simplified for a user. Safety of the injection device 1 may be improved. Unnecessary energy consumption by the electronic circuitry 20 may be reduced.

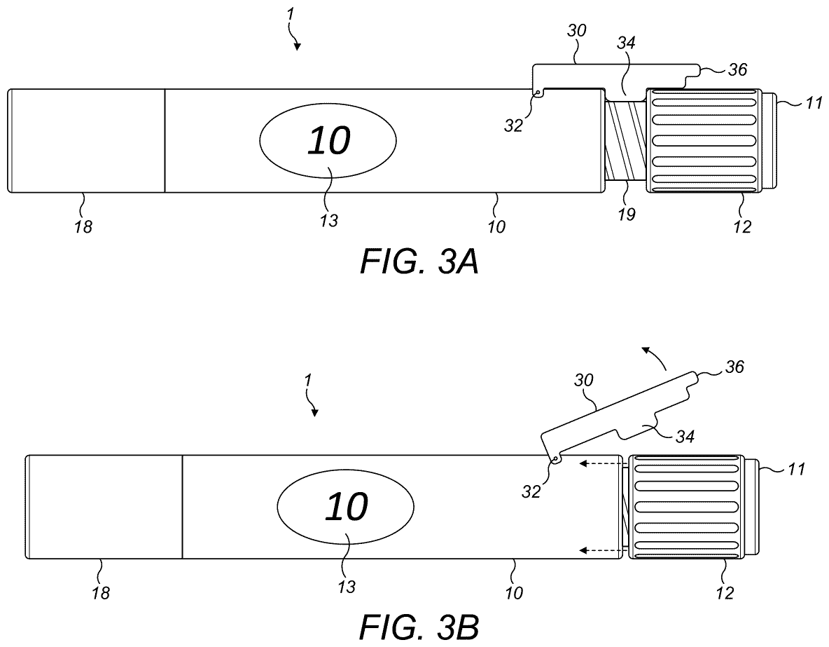

FIG. 3A and FIG. 3B show an injection device 1 according to a first embodiment. The injection device 1 is similar to the injection device 1 as described in relation to FIG. 1 and FIG. 2, however the injection device 1 also includes an actuation member. In this embodiment, the actuation member is formed as a hinged member 30, which may take the form of a clamp or a latch, for example. The hinged member 30 is pivotally coupled to the housing 10 of the injection device 1 by a hinge 32 or the like.

The pivotal coupling of the hinged member 30 to the remainder of the injection device 1 allows the hinged member 30 to be pivoted by a user between a first position and a second position relative to the housing 10 of the injection device 1. FIG. 3A shows the hinged member 30 in the first position while FIG. 3B shows the hinged member 30 in the second position relative to the housing 10. The pivoting direction of the hinged member 30 is indicated by the curved arrow, while translation of the dose dialling member 19 and dosage knob 12 towards the housing 10 is indicated by the dotted arrows.

In the first position, the hinged member 30 is coupled to the injection device 1 by the hinge 32 and by an engagement feature 34 of the clamp. While the hinged member 30 is in the first position, the engagement feature 34 is configured to inhibit the hinged member 30 from moving to the second position.

The hinged member 30 may comprise a protrusion 36 extending from the main body of the hinged member 30 that can assist the user in moving the hinged member 30 from the first position to the second position. The user may be able to insert part of their finger under the protrusion 36 and apply pressure to the protrusion 36 to cause the hinged member 30 to pivot about the hinge 32 from the first position to the second position.

One or more mechanical operations of the injection device 1 may be inhibited by the presence of the hinged member 30 in the first position, but may be performed when the hinged member 30 is in the second position, as discussed previously. For example, a user may be inhibited from programming a dose into the injection device 1 while the hinged member 30 is in the first position. This may be because the engagement feature 34, or another part of the hinged member 30, engages with a mechanical part of the injection device 1 such as a dose dialling element to inhibit movement of the mechanical part. A user may move the hinged member 30 from the first position to the second position so that a dose may be programmed into the injection device 1. Moving the hinged member 30 from the first position to the second position causes the engagement feature 34, or the other part of the hinged member 30 which engages with the mechanical part of the injection device 1, to disengage from the mechanical part of the injection device 1 so that the mechanical part can move. A dose can therefore be programmed into the injection device 1. The mechanical part may be the dosage knob 12 or the dose dialling member 19, for example.

In some examples, a user may be inhibited from dispensing a dose using the injection device 1 while the hinged member 30 is in the first position. This may be because the engagement feature 34, or another part of the hinged member 30, engages with a mechanical part of the injection device 1 such as a dose dispensing element to inhibit movement of the mechanical part. A user may move the hinged member 30 from the first position to the second position so that a dose may be dispensed from the injection device 1. Moving the hinged member 30 from the first position to the second position causes the engagement feature 34, or the other part of the hinged member 30 which engages with the mechanical part of the injection device 1, to disengage from the mechanical part of the injection device 1 so that the mechanical part can move. A dose can therefore be dispensed from the injection device. The mechanical part may be the dosage knob 12, the dose dialling member 19, or the injection button 11, for example.

By having one or more operations of the injection device 1 inhibited by the presence of the hinged member 30 in the first position but not in the second position, a user can move the hinged member 30 from the first position to the second position each time they intend to perform that operation with the injection device 1.

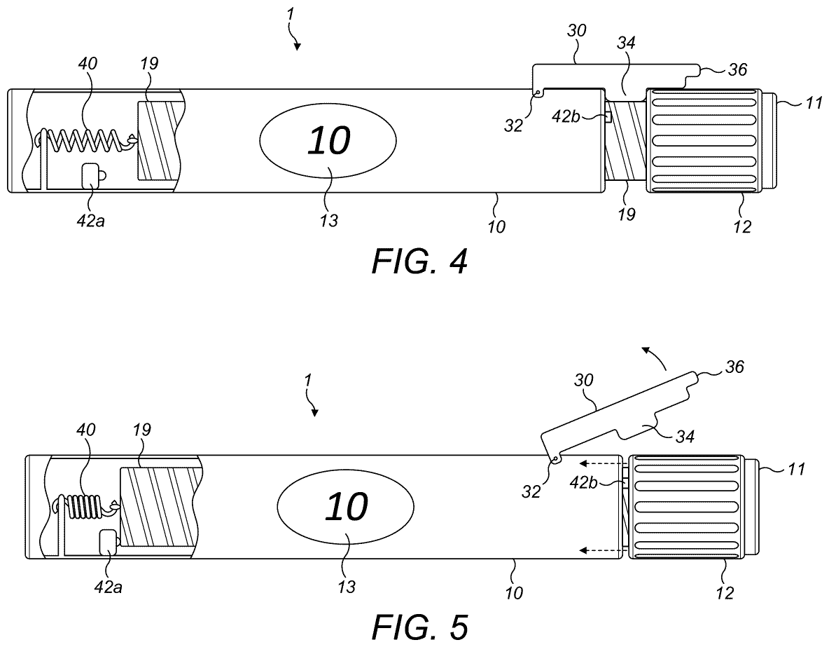

The actuation sensor arrangement 28 is arranged to detect when the hinged member 30 has moved from the first position to the second position. The actuation sensor arrangement 28 may take a number of forms, as exemplified in FIGS. 4 to 11 discussed below.

FIG. 4 shows a schematic side-view illustration of the injection pen 1 of FIG. 3A according to the first embodiment.

The hinged member 30 is movable between a first position and a second position as discussed previously. FIG. 4 shows the hinged member 30 in the first position while FIG. 5 shows the injection pen of FIG. 4 with the hinged member 30 in the second position.

FIG. 4 shows the dosage knob 12 separated a particular distance from the housing 10 of the injection device 1. This is a first position of the dosage knob 12 relative to the housing 10. The dosage knob 12 is able to translate relative to the housing 10, towards the housing 10. The dosage knob 12 is biased to move in a direction towards the housing 10 by a biasing member 40. The biasing member 40 may comprise a resilient member such as a spring, as shown in the cutaway portion of FIG. 4. It can be seen in FIG. 4 that the biasing member 40 is coupled between the housing 10 and the dose dialling member 19. The biasing member 40 acts on the dose dialling member 19 to bias the dose dialling member 19 towards the housing 10, in a direction parallel to the longitudinal axis of the housing 10. The dosage knob 12 is coupled to the dose dialling member 19 and so the dosage knob 12 is also biased towards the housing 10 by the biasing member 40.

In the example shown in FIG. 4, where the biasing member 40 comprises a spring, the spring is in a stretched state when the dosage knob 12 is in the first position relative to the housing 10. The spring tries to contract to its relaxed state, thereby exerting a force that attempts to pull the dosage knob 12 towards the housing 10.

FIG. 4 shows one example arrangement of the biasing means 40 being used to bias the dosage knob 12, however any other suitable arrangement may be used. For example, the biasing member 40 may be coupled between the proximal end of the housing 10 and the dosage knob 12, rather than via the dose dialling member 19.

The dosage knob 12 is held in its first position relative to the housing 10 by the hinged member 30, when the hinged member is in the first position. In this particular example, the dosage knob 12 is held in the first position by the engagement feature 35 being located between the housing 10 and the dosage knob 12.

In order to activate the injection device 1, a user moves the hinged member 30 from the first position to the second position. FIG. 5 shows injection device of FIG. 4 once the hinged member has been moved from its first position to its second position, as indicated by the curved arrow. Once the hinged member 30 has been moved from its first position to its second position, the dosage knob 12 is no longer held in its first position relative to the housing 10. The dosage knob 12 is now free to move further towards the housing 10 and into its second position relative to the housing 10. The dosage knob 12 automatically moves from its first position to its second position due to the bias applied by the biasing member 40. FIG. 5 shows the dosage knob 12 in its second position, whereby it has translated axially towards the housing 10. It can be seen in the cutaway segment of FIG. 5 that the dose dialling member 19 has also translated axially in concert with the dosage knob 12, further into the housing 10.

In the embodiment of FIG. 4 and FIG. 5, the actuation sensor arrangement 28 comprises a switch 42a, 42b. The switch may be a microswitch or the like. The switch 42a, 42b is arranged in the injection device 1 so that it can detect whether the dosage knob 12 is in its first position or its second position relative to the housing 10, thereby detecting whether the hinged member 30 is in its first position or second position relative to the housing 10. The switch 42a, 42b may be in first state (e.g. open) when the dosage knob 12 is in its first position and a different, second state (e.g. closed) when the dosage knob 12 is in its second position.

FIG. 4 shows two switches 42a, 42b arranged at different locations in the injection device 1. The locations shown illustrate two example positions for the switch 42a, 42b. Some injection devices use one of the switches 42a, 42b.

FIG. 4 and FIG. 5 show a first example location of the switch 42a. The switch 42a is located within the housing 10. The switch 42a is located so that it is not activated by the dose dialling member 19 when the dosage knob 12 is in its first position, as shown in FIG. 4, but is activated by the dose dialling member 19 when the dosage knob is in its second position, as shown in FIG. 5. The switch 42a may be in a closed state when activated and an open state when not activated, or may be in an open state when activated and a closed state when not activated. The switch 42a is therefore able to detect whether the dosage knob is in the first position or the second position. The electronic circuitry 20 of the injection device 1 may be activated in response to detection by the switch 42a that the dosage knob 12 is in the second position, and therefore that the hinged member 30 has been moved from its first position to its second position.

FIG. 4 and FIG. 5 both show an alternative location for a switch 42b. Here, the switch 42b is located at a proximal end of the housing 10 and is arranged to be activated by the dosage knob 12 when it moves from its first position to its second position. In other examples, the switch 42b may be located on the dosage knob 12 and may be arranged to be activated by the housing 10 when the dosage knob 12 moves into its second position. Any alternative suitable location for the switch 42a, 42b may be used.

A user may later move the dosage knob 12 from its second position back to its first position by pulling it away from the housing 10. The user may subsequently move the hinged member 30 from its respective second position to its first position in order to hold the dosage knob 12 in its first position. This movement of the dosage knob 12 from the second position to the first position may be detected by the switch 42a, 42b. In response to the detection, the electronic circuitry 20 may be moved from the activated state back into a dormant state.

In other examples, the dosage knob 12 may instead be biased away from the housing 10 of the injection device 1 instead of towards the housing 10. The second position of the dosage knob relative to the housing may therefore be further from the housing than the first position of the dosage knob 12 relative to the housing 10. The hinged member 30 may extend across the entire length of the dosage knob 12, with the engagement feature 34 being located against the proximal end of the dosage knob 12 when the hinged member 30 is in the first position. Therefore when the hinged member 30 is in its first position, it may hold the dosage knob 12 in its first position relative to the housing 10 and inhibit movement of the dosage knob 12 to the second position of the dosage knob 12 relative to the housing 10. When the hinged member 30 is moved from its first position to its second position, the dosage knob 12 is no longer held by the hinged member 30 and is free to move from its first position to its second position under the influence of the biasing member 40. A switch 42a, 42b is arranged in the injection device to detect this movement of the dosage knob 12 from the first position to the second position, so that the electronic circuitry 20 can be activated.

The aforementioned embodiment discussed in relation to FIG. 4 and FIG. 5 describes the dosage knob 12 being biased from the first position towards the second position. However in other examples, it is a different feature of the injection device 1 that is biased, rather than the dosage knob 12. For example, the injection button 11 may be biased by the biasing member 40.

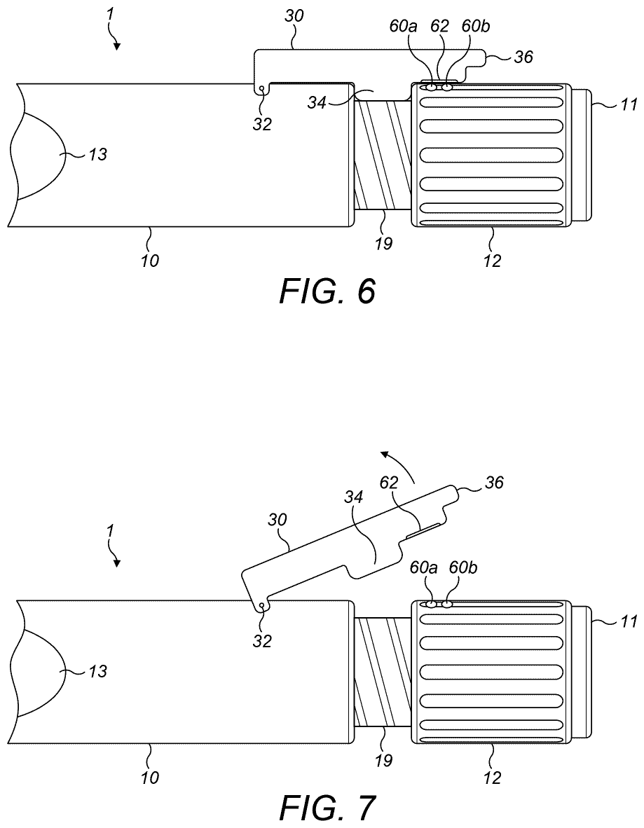

FIG. 6 shows an injection device 1 according to a second embodiment, in particular the proximal end of an injection device 1. The injection device is similar to the injection device 1 of FIG. 4 and FIG. 5, however in this embodiment the dosage knob 12 may not be biased towards the housing 10.

FIG. 6 shows the actuation sensor arrangement 28 comprising two electrical contacts 60a, 60b formed on an outer surface of the dosage knob 12. The hinged member 30 has an electrically conductive portion 62 formed on a surface which faces the electrical contacts 60a, 60b when the hinged member 30 is in its first position. The electrically conductive portion 62 is located on the hinged member 30 so that it forms an electrical contact with both of the electrical contacts 60a, 60b when the hinged member 30 is in the first position. The electrically conductive portion 62 may be formed from an electrically conductive wire, film, ink, strip or the like.

The two electrical contacts 60a, 60b and electrically conductive portion 62 effectively form a switch that is ‘closed’ when the hinged member 30 is in the first position, as shown in FIG. 6. A current is therefore able to flow from the first electrical contact 60a to the second electrical contact 60b via the electrically conductive portion 62 when the hinged member 30 is in the first position.

In order to activate the injection device 1, a user moves the hinged member 30 from the first position to the second position. FIG. 7 shows the injection device 1 of FIG. 6 after the hinged member 30 has been moved from the first position to the second position, as indicated by the curved arrow. The electrically conductive portion 62 is no longer in electrical contact with both of the electrical contacts 60a, 60b, and so a current is no longer able to flow from the first electrical contact 60a to the second electrical contact 60b. The switch effectively formed by the two electrical contacts 60a, 60b and electrically conductive portion 62 is therefore ‘open’.

The injection device 1 is activated in response to a detection by the two electrical contacts 60a, 60b that the hinged member 30 has moved from the first position to the second position. In this case, it is detected that the electrical contacts 60a, 60b have changed from being in electrical contact via the electrically conductive portion 62 to no longer being in electrical contact via the electrically conductive portion 62, thereby indicating that the hinged member 30 has moved from the first position to the second position.

In some examples it is the processor arrangement 23 that determines that a current is no longer able to flow from the first electrical contact 60a to the second electrical contact 60b and in response activates the electronic circuitry 20. In other examples, the switch effectively formed by the two electrical contacts 60a, 60b and electrically conductive portion 62 short-circuits a connection between the power supply 29 and one or more components of the electronic circuitry 20 when the hinged member 30 is in its first position. However, when the hinged member 30 is in its second position, the connection between the power supply 29 and the one or more components is no longer short-circuited by the two electrical contacts 60a, 60b and electrically conductive portion 62, allowing power to be supplied to the one or more components of the electronic circuitry 20 from the power supply 29.

FIGS. 6 and 7 show the electrical contacts 60a, 60b both formed on the outer surface of the dosage knob 12. However in other examples the electrical contacts 60a, 60b may be located elsewhere on the injection device 1, for example on the dose dial sleeve 19, the housing 10 or the injection button 11. In some examples, the electrical contacts 60a, 60b may both be formed on the hinged member 30 while the electrically conductive portion 62 is located on the dosage knob 12, the dose dial sleeve 19, the housing 10 or the injection button 11.

After moving the hinged member 30 from the first position to the second position, a user may subsequently move the hinged member 30 back from its second position to its first position, for example after an injection has been completed. This action reconnects the first electrical contact 60a and the second electrical contact 60b via the electrically conductive portion 62. This reconnection may be detected by the processor arrangement 23. In response to the detection, the electronic circuitry 20 may be placed from the activated state back into a dormant state.

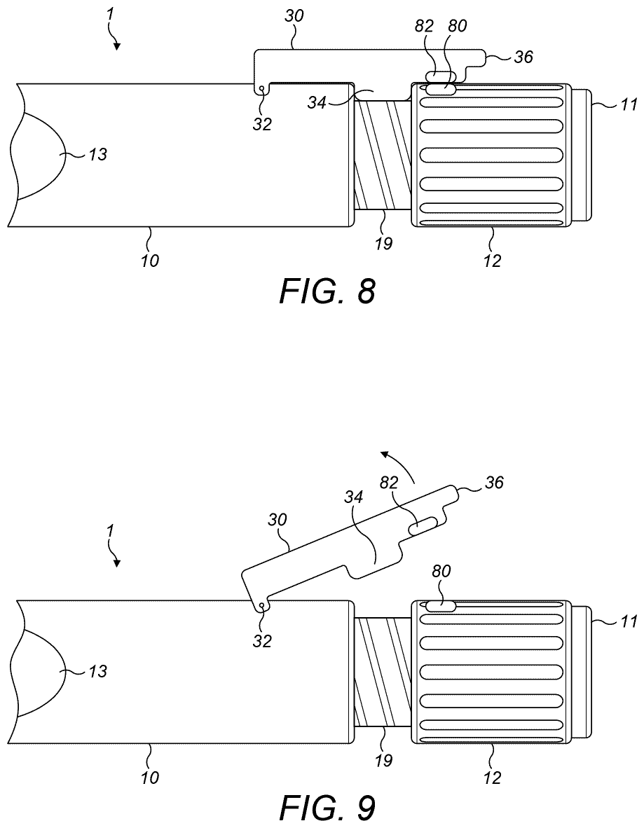

FIG. 8 shows an injection device 1 according to a third embodiment. The injection device 1 is similar to the injection device of FIG. 6, however the actuation sensor arrangement 28 instead comprises a reed switch 80 located in the dosage knob 12. The reed switch 80 is movable between an open and closed state. The reed switch 80 may normally be in an open state but move to a closed state in the presence of a magnet 82. Alternatively, the reed switch 80 may normally be in an open state but moved to a closed state in the presence of a magnet 82.

A magnet 82 is located in the hinged member 30, for example proximate a surface of the hinged member 30 that is adjacent to an outer surface of the injection device 1 when the hinged member 30 is in its first position. The reed switch 80 and magnet 82 are each located so that the magnet 82 interacts with the reed switch 80 when the hinged member 30 is in the first position, but not when it is in the second position.

To activate the injection device 1, a user moves the hinged member 30 from the first position to the second position. FIG. 9 shows the injection device 1 of FIG. 8 once the hinged member 30 has been moved into the second position, as indicated by the arrow. As the hinged member 30 is moved from the first position to the second position, the magnet 82 is moved away from the reed switch 80. When the hinged member 30 is in the first position the magnet 82 interacts with the reed switch 80 to maintain it in a first switch state (either open or closed). However when the hinged member 30 is moved into the second position, the magnet 82 has comparatively little or no interaction with the reed switch 80, resulting in the reed switch moving to a second switch state (closed or open).

The injection device 1 is activated in response to a detection by the reed switch 80 that the hinged member 30 has moved from the first position to the second position. In this case, it is detected that the reed switch 80 has changed from closed state to an open state (or open state to a closed state), thereby indicating that the hinged member 30 has moved from the first position to the second position. In response to the detection, the injection device 1 is activated, as described previously.

A user may subsequently move the hinged member 30 back from its second position to its first position, thereby bringing the magnet 82 back into proximity of the reed switch 80 and causing the reed switch 80 to be activated (i.e. moved from a closed state to an open state or vice versa). In response to the detection by the reed switch 80 that the hinged member 30 is back in its first position, the electronic circuitry 20 may be moved from the activated state back into a dormant state. This may be performed by the processor arrangement 23.

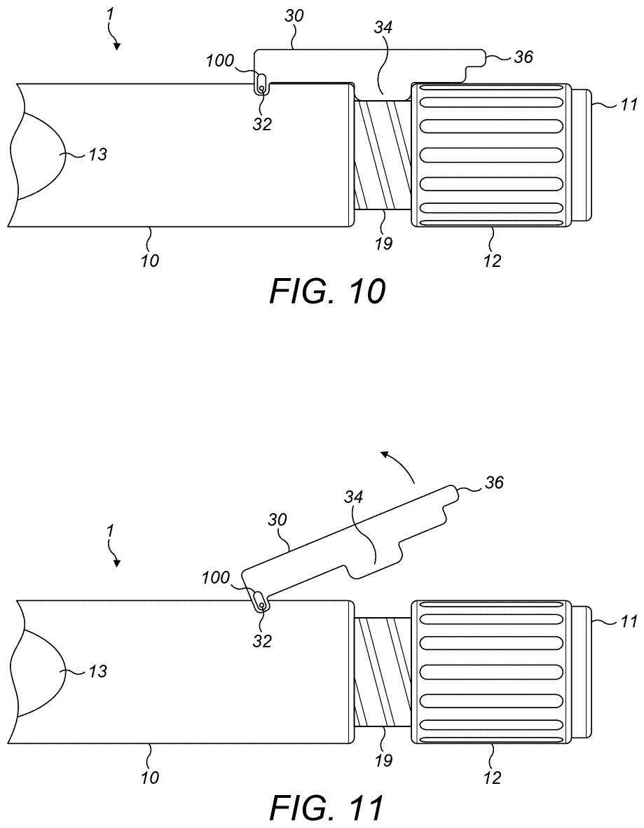

FIG. 10 and FIG. 11 show an injection device 1 according to a fourth embodiment. The injection device 1 is similar to the injection device of FIG. 6, however the actuation sensor arrangement 28 instead comprises a hinge switch 100. The hinge switch 100 is located proximate to, or forms part of, the hinge 32. The hinge switch 100 is movable between a first switch state and a second switch state. The hinge switch 100 is in the first switch state when the hinged member 30 is in the first position relative to the housing 10, as shown in FIG. 10. The hinge switch 100 is in the second switch state when the hinged member 30 is in the second position relative to the housing 10, as shown in FIG. 11. The first switch state may be an open switch state while the second switch state is a closed switch state. However, in other examples the first switch state may be a closed switch state while the second switch state is an open switch state.

FIG. 10 shows the hinged member 30 in the first position and the hinge switch 100 in the first switch state. In order to activate the injection device 1, a user moves the hinged member 30 from the first position to the second position. FIG. 11 shows the hinged member 30 now in the second position after being rotated in a direction as illustrated by the arrow, with the hinge switch 100 now in the second switch state.

The injection device 1 is activated in response to a detection that the hinge switch 100 has moved from the first switch state to the second switch state. The injection device 1 may be activated as described previously in relation to other embodiments.

A user may subsequently move the hinged member 30 back from its second position to its first position. The movement of the hinged member 30 from the second position to the first position can be detected by the hinge sensor 100. In response to the detection, the processor arrangement 23 may move the electronic circuitry 20 from the activated state back into a dormant state.

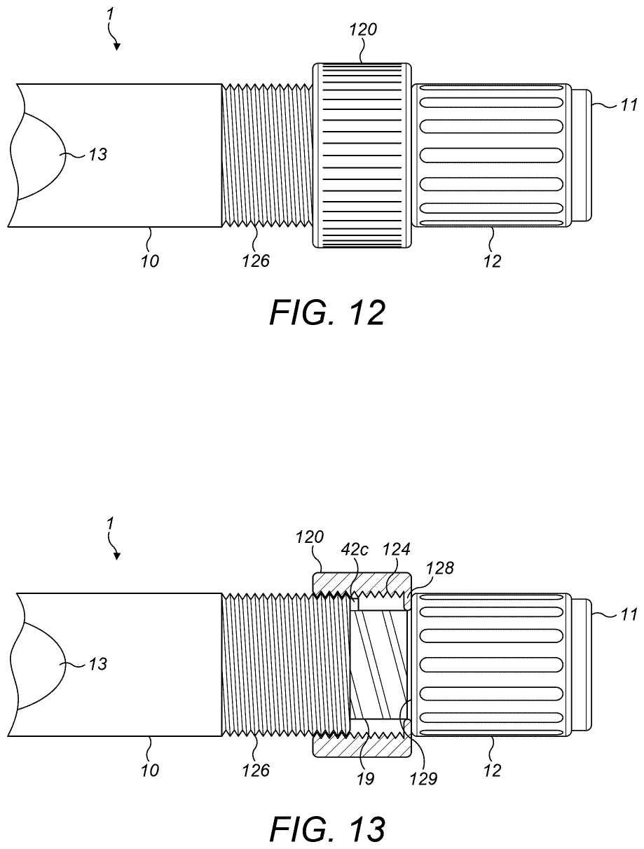

FIG. 12 shows an injection device 1 according to a fifth embodiment. The injection device 1 partially shown in FIG. 12 is similar to the injection device 1 previously described in relation to FIG. 3, however the actuation member of the injection device 1 comprises a collar 120 instead of a hinged member 30.

The collar 120 is located concentrically around the cylindrical housing 10 of the injection device 1, at a proximal end of the housing 10. The collar 120 is therefore located at the same end of the housing 10 as the dosage knob 12.

FIG. 13 shows the same injection device 1 of FIG. 12, however the collar 120 is shown in cross-section for clarity. The collar 120 has a thread 124 formed on an inner surface of the collar 120. The thread 124 is configured to interact with a corresponding thread 126 formed on the outer surface of the housing 10. The thread 124 of the collar 120 and the thread 126 of the housing are arranged to interact such that rotation of the collar 120 around the housing 10 causes the collar 120 to translate axially along the longitudinal axis of the housing 10.

Rotation of the collar 120 in a first rotational direction around the longitudinal axis of the housing 10 and relative to the housing 10 causes the collar 120 to translate axially along the longitudinal axis of the housing 10 in a first axial direction, for example from the proximal end towards the distal end of the housing 10. Conversely, rotation of the collar 120 in a second rotational direction opposite the first rotational directions around the longitudinal axis of the housing 10 and relative to the housing 10 causes the collar 120 to translate axially along the longitudinal axis of the housing 10 in a second axial direction opposite the first axial direction, for example from the distal end towards the proximal end of the housing 10.

FIG. 13 shows the collar 120 having a flange 128 located at a proximal end of the collar 120. The flange 128 is arranged such that it extends between the housing 10 and the dosage knob 12, protruding radially towards the center of the aperture formed by the collar 120. The flange 128 is arranged to prevent the dosage knob 12 from moving through the collar 120. The flange 128 therefore limits movement of the dosage knob 12 towards the housing 10. The dosage knob 12 can translate towards the housing 10 until a distal surface 129 of the dosage knob 12 makes contact with the flange 128, after which further movement of the dosage knob 12 towards the housing 10 is inhibited.

In some examples, a diameter of the dosage knob 12 may be larger than the diameter of the aperture of the collar 120, thereby preventing the dosage knob 12 from moving through the collar 120. A flange 128 may therefore not be required on the collar 120.

One or more mechanical operations of the injection device 1 may be inhibited by the presence of the collar 120 in the first position but may be performed when the collar 120 is in the second position, in a similar manner as described in the embodiment of FIG. 3A. For example, the presence of the collar 120 in the first position may inhibit the dosage knob 12 being used for a dose dialling operation and/or a dose dispensing operation. If the collar 120 is moved to the second position of the collar 120, the dose dialling operation and/or a dose dispensing operation may now be permitted.

The dosage knob 12 may be biased towards the housing 10 from a first position relative to the housing 10 towards a second position relative to the housing 10, as previously described in relation to FIG. 4 and FIG. 5.

In order to activate the injection device 1, a user moves the collar 120 from a first position relative to the housing 10 to a second position relative to the housing 10 by rotating the collar 120 as discussed previously. The rotation of the collar 120 relative to the housing 10 causes it to translate along the housing 10 from a proximal end towards a distal end of the housing 10. The first and second positions of the collar 120 relative to the housing 10 are therefore linear positions relative to the housing 10, but could be rotational positions.

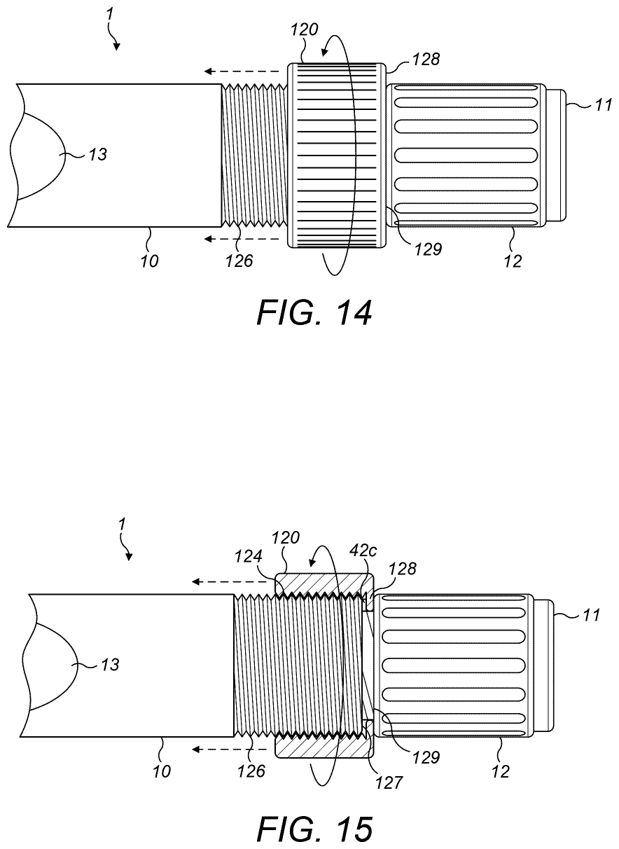

FIG. 14 shows the injection device 1 of FIG. 12 once the collar 120 has been moved into the second position by a user rotating the collar 120. FIG. 15 shows the injection device 1 of FIG. 14, however with the collar 120 shown in cross-section for convenience. It can be seen in FIG. 14 and FIG. 15 that the collar 120 has been translated along the longitudinal axis of the injection device 1, along the length of the housing 10. The bias of the dosage knob 12 towards the housing 10 has led to the dosage knob 12 also moving towards the housing 10 in concert with the collar 120 from a first position to a second position of the dosage knob 12 relative to the housing 10. The dosage knob 12 abuts the flange 128, but can move no further towards the housing 10 due to the presence of the flange 128.

FIG. 12 and FIG. 13 show the dosage knob 12 in the first position relative to the housing 10, while FIG. 14 and FIG. 15 show the dosage knob 12 in the second position relative to the housing 10. The actuation sensing arrangement 28 is configured to detect whether the collar 120 has moved from its first position to the second position by detecting whether the dosage knob 12 has moved from its first position to the second position. The injection device 1 is activated in response to detection by the actuation sensing arrangement 28 that the dosage knob 12 has been moved from its first position to its second position.

Similar to the embodiment described in relation to FIG. 4 and FIG. 5, the actuation sensing arrangement 28 may comprise a switch 42a, 42b which is activated by the dosage knob 12 moving into its second position due to the biasing member 400

In other examples, the actuation sensing arrangement 28 comprises a switch 42c arranged to be directly activated by the collar 120. FIG. 13 and FIG. 15 show the switch 42c located at a surface 127 at the proximal end of the housing 10. The switch 42c is arranged to be in a first state (open or closed) when the collar 120 is in its first position along the housing 10, as shown in FIG. 13. However, the switch 42c is arranged to be moved into a second, different state (closed or open) once the collar 120 has been moved into its second position along the housing 10, as shown in FIG. 15. FIG. 15 shows the switch 42c being pressed by the flange 128 of the collar 120 while the collar is in its second position, however the switch 42c may alternatively be pressed by a different part of the collar 120.

The switch 42c is therefore able to detect whether the collar 120 has moved from its first position into its second position. In such an example, it is not necessary that the dosage knob 12 is biased towards the housing 10. This means that the dosage knob 12 may remain in its first position relative to the housing 10 as the collar 120 is moved from its first position to its second position.

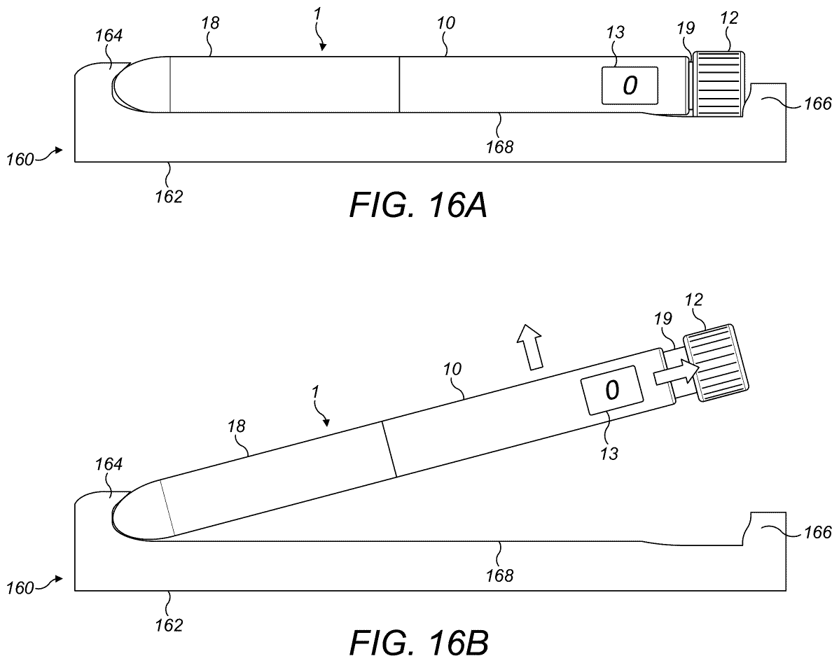

FIG. 16A and FIG. 16B show an injection device 1 according to a sixth embodiment. The injection device 1 shown in FIG. 16A and FIG. 16B is similar to the injection device described in relation to FIG. 1 and FIG. 2, however the injection device 1 now includes an actuation member formed as a container 160. Removal, or at least partial removal, of the injection device 1 from the container 160 causes activation of the electronic circuitry 20 within the injection device 1. Aspects of this embodiment may be particularly suitable for one-time use injection devices 1 such as autoinjectors and injection devices 1 that would not be placed back into the container 160 after removal.

The container 160 is configured to hold the injection device 1. FIG. 16A shows the injection device 1 when held in the container 160, while FIG. 16B shows the injection device 1 of FIG. 16A when partially removed from the container 160.

The container 160 is formed from a main body 162, a first retaining feature 164 and a second retaining feature 166. The first retaining feature 164 and second retaining feature 166 each extend upwards from the main body 162 of the container 160 to form a recess 168 therebetween. The recess 168 is dimensioned to at least partially contain the injection device 1.

The injection device 1 of FIG. 16A and FIG. 16B is similar to the injection device 1 previously described in relation to FIG. 4 in that the dosage knob 12 is biased relative to the housing 10. However in the embodiment of FIG. 16A and FIG. 16B, the dosage knob 12 is biased in a direction away from the housing 10 rather than towards the housing 10. The biasing may be facilitated using a resilient member or the like as discussed previously, or any other suitable means.

FIG. 16A shows the dosage knob 12 in a first position relative to the housing 10. FIG. 16B shows the dosage knob in a second position relative to the housing 10. The second position of the dosage knob 12 is more distant from the housing 10 than the first position of the dosage knob 12. The dosage knob 12 is biased from the first position towards the second position. When the dosage knob 12 moves from the first position to the second position, the dosage knob 12 translates away from the housing 10, in a direction parallel to the longitudinal axis of the housing 10.

The first retaining feature 164 and second retaining feature 166 of the container 160 are separated by a distance that allows the injection device 1 to be held between the first retaining feature 164 and a second retaining feature 166 when the dosage knob 12 is in its first position relative to the housing 10, but not when the dosage knob 12 is in its second position relative to the housing 10. This is because the injection device 1 has a shorter longitudinal length when the dosage knob 12 is in the first position comparted to when it is in the second position.

FIG. 16A shows the dosage knob 12 in the first position and so the injection device 1 is held between the first retaining feature 164 and the second retaining feature 166 of the container 160. The second retaining feature 166 contacts the dosage knob 12 and/or the injection button 11 while the first retaining feature 164 contacts a feature at the distal end of the injection device 1, such as the distal end of the cap 18. The dosage knob 12 is biased from the first position towards the second position, thereby exerting a force on the first retaining feature 164 and on the second retaining feature 166. The forces exerted on the first retaining feature 164 and the second retaining feature 166 may hold the injection device 1 in position in the container 160.

In order to activate the injection device 1, a user at least partially removes the injection device 1 from the container 160. FIG. 16B shows the injection device 1 of FIG. 16A after it has been partially removed from the container 160 by a user. The user has lifted the injection device 1 away from the container 160. As the proximal end of the injection device 1 is moved away from the container 160, the dosage knob 12 is brought out of contact with the second retaining feature 166. The dosage knob 12 can no longer apply a force to the second retaining feature 166 and so the biasing of the dosage knob 12 away from the housing 10 causes the dosage knob 12 to move from the first position to the second position relative to the housing 10. FIG. 16B shows the dosage knob 12 in the second position relative to the housing 10.

The injection device 1 contains an actuation sensing arrangement 28 configured to detect when the dosage knob 12 has moved from its first position relative to the housing 10 to its second position relative to the housing 10, and therefore that the injection device 1 has been at least partially removed from the container 160. The actuation sensing arrangement may comprise a switch 42a, 42b, 42c, for example as described previously in relation to other embodiments. The switch 42a, 42b, 42c is arranged in the injection device to be closed when the dosage knob 12 is in its first position and open when the dosage knob 12 is in its second position, or vice versa. The switch 42a, 42b, 42c therefore detects whether the dosage knob 12 is in the first positon or the second position. In response to a detection that the dosage knob 12 has moved from its first position to its second position, the electronic circuitry 20 may be activated.

In some examples, the electronic circuitry 20 of the injection device 1 may be put back into a dormant state by placing the injection device 1 back in the container 160. The process is similar to that described previously with reference to FIG. 16A and FIG. 16B but in reverse. The dosage knob 12 starts in the second position relative to the housing 10 when the injection device 1 is not in the container 160. However, when a user places the injection device 1 in the container 160 between the first retaining feature 164 and the second retaining feature 166, they move the dosage knob 12 from the second position to the first position. This may be detected by a sensor 42a, 42b, 42c as discussed previously. In response to detecting that the dosage knob 12 has moved from the second position to the first position, the electronic circuitry 20 may be placed in a dormant state.

The injection device 1 being held in the container 160 as shown in FIG. 16A may be considered the first position of the container 160 relative to the housing 10 of the injection device 1. At least one mechanical operation of the injection device may be inhibited while the container 160 is in its first position, as discussed previously. In this example, the container 160 is preventing movement of the dosage knob 12 and so is preventing both a dose programming operation and a dose dispensing operation from taking place. The at least partial removal of the container 160 from the injection device 1 as shown in FIG. 16B may be considered the second position of the container 160 relative to the housing 10 of the injection device 1. The at least one mechanical operation of the injection device may now no longer be inhibited while the container 160 is in its second position, as discussed previously. In this example, the container 160 is no longer preventing movement of the dosage knob 12 and so is no longer preventing both a dose programming operation and a dose dispensing operation from taking place.

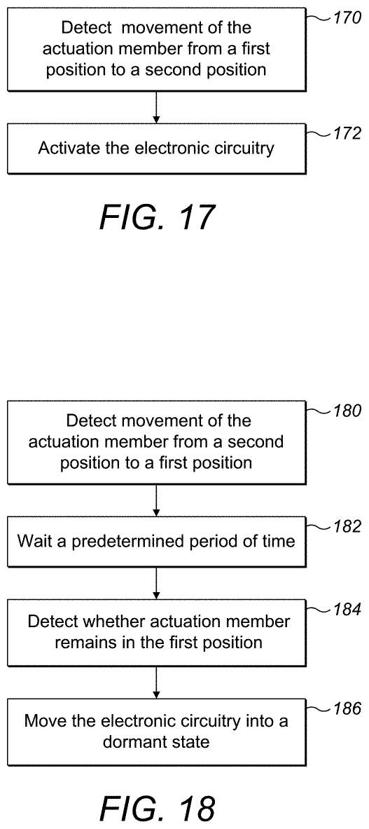

FIG. 17 is a flowchart illustrating a method of activating an injection device 1 according to any of the aforementioned embodiments. The method is performed by the actuation sensing arrangement 28 and the processor arrangement 23, for example.

In step 170, the movement of an actuation member from a first position relative to the housing 10 of the injection device 1 to a second position relative to the housing 10 of the injection device 1 is detected by the actuation sensing arrangement 28. The actuation member may have been moved from the first position to the second position by a user of the injection device 1.

In step 172, and in response to detection of the movement of the actuation member form the first position to the second position, the electronic circuitry 20 is activated as discussed previously.

In some examples, step 172 is performed as soon as it is detected by the actuation sensing arrangement 28 that the actuation member has moved from its first position relative to the housing 10 of the injection device 1 to its second position relative to the housing 10 of the injection device 1. However, in other examples step 172 is performed a predetermined period of time after the detection by the actuation sensing arrangement 28. The predetermined period of time may be a period of time between 1 second and 110 seconds, for example.

In some examples, after the predetermined period of time has elapsed, it is again detected whether the actuation member remains is in its second position. If the actuation member has remained in its second position, step 172 is performed. However, if the actuation member no longer remains is in its second position then step 172 is not performed and the electronic circuitry remains in its dormant state. In some examples, step 172 may only be performed if it has been detected that the actuation member remains in its second position during the entirety of the predetermined time period.

FIG. 18 is a flowchart illustrating a further method that can be carried out by the injection device 1 of any of the previous embodiments. The method steps of FIG. 18 may be carried out after method step 172 of FIG. 17.

In step 180, the movement of the actuation member from the second position relative to the housing 10 to the first position relative to the housing 10 is detected by the actuation sensing arrangement 28.

In step 182, the electronic circuitry 20 waits for a predetermined period of time to elapse in response to the detection by the actuation sensing arrangement 28. The predetermined period of time may be between 1 second and 10 seconds, such as 3 seconds, for example.

In step 184, after the predetermined period of time has elapsed, it is detected whether the actuation member remains in the first position. If so, the method moves to step 186 in which the electronic circuitry 20 is moved from the active state to a dormant state. If not, the electronic circuitry 20 remains in the active state. In some examples, the actuation member remains in the first position during the entire predetermined period of time for the electronic circuitry 20 to be placed into the dormant state.

In some examples, step 184 is not present. The method therefore moves from step 182 straight to step 186. By waiting a predetermined period of time before moving the electronic circuitry 20 into a dormant state, the processor arrangement 23 is given additional time to finish perform any currently running functions.

In some examples, both step 180 and step 184 are not present. In such a case, the method moves straight from step 182 to step 186. Therefore the electronic circuitry 20 is moved into the dormant state almost immediately in response to detection that the actuation member has moved from its first position to its second position.

The above embodiments have generally been described with the injection device 1 being pen injector. However, the above embodiments can also be used with other types of injection devices including, but not limited to, autoinjectors and patch pumps.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An example of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

1-15. (canceled)

16. An injection device comprising:
a housing;
an actuation member movable between a first position relative to the housing and a second position relative to the housing, the actuation member being arranged to inhibit a mechanical operation of the injection device when the actuation member is in the first position and allow the mechanical operation of the injection device when the actuation member is in the second position;
an actuation sensing arrangement configured to detect movement of the actuation member from the first position relative to the housing to the second position relative to the housing; and
electronic circuitry configured to be activated in response to detection by the actuation sensing arrangement that the actuation member has moved from the first position relative to the housing to the second position relative to the housing.

17. The injection device according to claim 16, wherein the mechanical operation comprises a dose programming operation.

18. The injection device according to claim 16, wherein the mechanical operation comprises a dose dispensing operation.

19. The injection device according to claim 16, wherein activation of the electronic circuitry comprises switching the electronic circuitry from a low-power state to a high-power state.

20. The injection device according to claim 16, wherein the actuation member comprises a hinged member pivotally coupled to the housing such that the hinged member can pivot between the first position and the second position relative to the housing.

21. The injection device according to claim 20, wherein the actuation sensing arrangement comprises a plurality of conductive contacts, and the hinged member comprises an electrically conductive portion.

22. The injection device according to claim 21, wherein the conductive contacts and the electrically conductive portion are arranged such that:
the electrically conductive portion forms an electrical connection between the conductive contacts when the actuation member is in one of the first position and the second position; and
there is no electrical connection between the conductive contacts via the electrically conductive portion when the actuation member is in the other one of the first position and the second position.

23. The injection device according to claim 20, wherein the actuation sensing arrangement comprises a hinge switch arranged to detect whether the hinged member is in the first position or the second position.

24. The injection device according to claim 20, wherein the actuation sensing arrangement comprises a reed switch, and the hinged member comprises a magnet.

25. The injection device according to claim 24, wherein the magnet and the reed switch are arranged such that the reed switch is in a first switch state when the actuation member is in the first position and the reed switch is in a second switch state when the actuation member is in the second position.

26. The injection device according to claim 20, further comprising a dosage knob that is biased from a first linear position relative to the housing towards a second linear position relative to the housing, the hinged member being configured to inhibit movement of the dosage knob from the first linear position to the second linear position when the hinged member is in the first position, and the dosage knob being configured to move from the first linear position to the second linear position in response to the hinged member being moved from the first position to the second position.

27. The injection device according to claim 26, wherein the actuation sensing arrangement is configured to detect movement of the dosage knob from the first linear position relative to the housing to the second linear position relative to the housing, and the electronic circuitry is configured to be activated in response to the detection by the actuation sensing arrangement that the dosage knob has moved from the first linear position relative to the housing to the second linear position relative to the housing.

28. The injection device according to claim 16, wherein the actuation member comprises a container configured to hold the injection device, the actuation sensing arrangement is configured to detect removal of the injection device from the container, and the electronic circuitry is configured to be activated in response to detection by the actuation sensing arrangement that the injection device has been removed from the container.

29. The injection device according to claim 16, wherein the actuation member comprises a collar threadingly engaged with the housing of the injection device, the collar being movable along the housing from the first position relative to the housing to the second position relative to the housing.

30. The injection device according to claim 29, wherein the actuation sensing arrangement is configured to detect movement of the collar from the first position relative to the housing to the second position relative to the housing, and the electronic circuitry is configured to be activated in response to detection by the actuation sensing arrangement that the collar has moved from the first position relative to the housing to the second position relative to the housing.

31. The injection device according to claim 16, wherein the actuation sensing arrangement is further configured to detect movement of the actuation member from the second position relative to the housing to the second position relative to the housing, and the electronic circuitry is configured to be switched from an activated state to a dormant state in response to the detection by the actuation sensing arrangement that the actuation member has moved from the second position relative to the housing to the first position relative to the housing.

32. The injection device according to claim 16, further comprising a container containing a medicament.

33. The injection device according to claim 16, wherein the injection device is an injection pen or a patch pump.

34. A method comprising:
detecting, by an actuation sensing arrangement of an injection device, movement of an actuation member of the injection device from a first position relative to a housing of the injection device to a second position relative to the housing, the actuation member being arranged to inhibit a mechanical operation of the injection device when the actuation member is in the first position and allow the mechanical operation of the injection device when the actuation member is in the second position; and
activating electronic circuitry of the injection device in response to the detected movement that the actuation member has moved from the first position relative to the housing to the second position relative to the housing.

35. The method of claim 34, wherein the mechanical operation comprises a dose programming operation or a dose dispensing operation.
